(19) United States
(12) Patent Application Publication
(10) Pub. No.: US 2012/0231073 A1
(43) Pub. Date: Sep. 13, 2012

(54) DEXLANSOPRAZOLE COMPOSITIONS

(75) Inventors: Nagaraju Manne, Hyderabad (IN); Udaykumar Neelam, Hyderabad (IN); Sudhakar Reddy Baddam, Hyderabad (IN); Naveen Kumar Kolla, Hyderabad (IN); Venkata Nookaraju Sreedharala, Hyderabad (IN); Chandra Sekhar Veera Venkata Naga Bulusu, Hyderabad (IN)

(73) Assignees: DR. REDDY'S LABORATORIES, INC., Bridgewater, NJ (US); DR. REDDY'S LABORATORIES LIMITED, Hyderabad (IN)

(21) Appl. No.: 13/475,565

(22) Filed: May 18, 2012

Related U.S. Application Data
(63) Continuation of application No. 12/426,537, filed on Apr. 20, 2009, now abandoned.
(60) Provisional application No. 61/081,538, filed on Jul. 17, 2008.

(30) Foreign Application Priority Data
Apr. 21, 2008 (IN) 965/CHE/2008

(51) Int. Cl. A61K 31/4439; A61P 1/04; A61K 9/52; A61K 9/14; A61K 9/48

(52) U.S. Cl. 424/457; 514/338; 424/400; 424/451; 424/463

(57) ABSTRACT

Premixes of dexlansoprazole with pharmaceutical excipients, processes for preparing premixes, pharmaceutical formulations containing the premixes, and their use in treatment of erosive esophagitis and heartburn associated with non-erosive gastroesophageal reflux disease.

### INTRODUCTION

The present invention relates to dexlansoprazole premixes with pharmaceutical excipients, pharmaceutical formulations containing the premixes, and processes for preparing the same. The invention further relates to therapeutic uses and methods of treatment employing such premix compositions.

Several substituted benzimidazole derivatives including rabeprazole, omeprazole, esomeprazole, lansoprazole, leminoprazole, pantoprazole, and mixtures thereof, are known to be useful for inhibiting gastric acid secretion in mammals and man by controlling gastric acid secretion at the final step of the acid secretory pathway. These active ingredients are acid-labile, creating several problems in formulating such acid-labile compounds into oral pharmaceutical dosage forms because of the acidic environment of the stomach, and have poor stability. In particular, they would be rapidly decomposed and change color under moist conditions or in an acidic to neutral aqueous solution.

When these compounds are formulated into pharmaceutical preparations for oral administration, they require special techniques to avoid contact of drug with gastric acid of the stomach. One technique most commonly used is to coat acid-labile compound, or its granules or pellets, with an enteric coating, which is insoluble in water under acidic conditions and soluble in water under neutral to alkaline conditions. However, the material used in enteric coatings itself is acidic, which can cause the decomposition of the acid-labile compound. Such decomposition occurs even during the enteric coating process, which results in the coloration of the surface of the drug-containing core. In order to avoid such problems, an inert subcoating, which is not acidic, is often required between the core and enteric coating, which increase the complexity and the cost of the formulation manufacture processes involving acid-labile compounds.

For substances that are labile in acid media, but have better stability in neutral to alkaline media, it is often advantageous to add alkaline reacting inactive constituents in order to increase the stability of the active compound during manufacture and storage. In particular, substituted benzimidazole derivatives such as omeprazole and esomeprazole are not only unstable in acidic conditions but also are not stable in the neutral solid state. Thus, in order to enhance the storage stability, an alkaline base such as sodium bicarbonate is added to the formulation, and/or the substituted benzimidazole derivatives are converted to their alkaline salts, which are usually more stable than the free species. It is also known that alkaline bases can have adverse effects on patients who suffer hypertension, heart failure, etc.

The active compound of the compositions of the present invention and methods is an optical isomer of the drug compound lansoprazole. Its chemical name is (+)-2-[(R)-{[3-methyl-4-(2,2,2-trifluoroethoxy)pyridin-2-yl]methyl}sulfinyl]-1H-benzimidazole, hereinafter referred to by the adopted name “dexlansoprazole,” and it has structural Formula I.

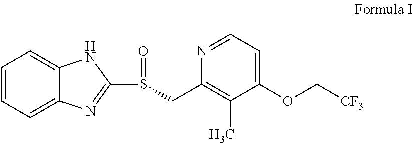

Dexlansoprazole is approved for marketing in the U.S.A. and it is presently commercially available in products with the brand name KAPIDEX® as 30 mg and 60 mg dual delayed release capsules, sold by Takeda Pharmaceuticals North America, Inc. The inactive excipients of KAPIDEX capsules include sugar spheres, magnesium carbonate, sucrose, low-substituted hydroxypropyl cellulose, titanium dioxide, hydroxypropyl cellulose, hypromellose 2910, talc, methacrylic acid copolymer, polyethylene glycol 8000, triethyl citrate, polysorbate 80, and colloidal silicon dioxide. The capsule shell is made of hypromellose, carrageenan and potassium chloride. Blue capsule shells contain FD&C Blue No. 2 and aluminum lake, gray capsule shells contain ferric oxide and aluminum lake, and both contain titanium dioxide. Dexlansoprazole has been approved for treating erosive esophagitis and heartburn associated with non-erosive gastroesophageal reflux disease (GERD).

U.S. Pat. Nos. 6,462,058 and 6,664,276 disclose crystalline forms of dexlansoprazole or a salt thereof.

U.S. Pat. Nos. 4,628,098, 4,786,505, 4,853,230, 5,689,333, 5,045,321, 5,093,132, and 5,433,959, of which the entire content is incorporated by reference, teach various stabilizing agents for their disclosed benzimidazole derivatives in core tablets. These patents also show that such compounds are stable in the presence of basic inorganic salts of magnesium, calcium, potassium and sodium. The stability is further consolidated by separating acid labile benzimidazoles from the acidic components of the enteric coating by interposing an intermediate coating (subcoating).

U.S. Pat. No. 6,013,281, of which the entire content is incorporated by reference, also discloses that a separating layer is formed in situ by direct application of an acidic enteric material onto an alkaline core containing benzimidazoles.

U.S. Patent Application Publication No. 2006/0057195 A1 describes stable solid preparations for medicinal use containing amorphous benzimidazole compounds including dexlansoprazole, which are produced by blending an amorphous benzimidazole compound with a nontoxic base such as a basic inorganic salt.

There remains a need for stable pharmaceutical compositions comprising dexlansoprazole or a pharmaceutically acceptable salt thereof.

### SUMMARY

The present invention relates to premix compositions comprising dexlansoprazole or a pharmaceutically acceptable salt thereof, pharmaceutical formulations containing the premixes, and methods of preparing the same.

In an embodiment, the invention includes premixes for use in pharmaceutical formulations of dexlansoprazole, said premixes comprising in combination: (a) dexlansoprazole or a pharmaceutically acceptable salt thereof; and (b) a water-soluble excipient.

In another embodiment, a premix further comprises a pharmaceutically acceptable basic compound, which can act as a stabilizer for the drug.

In an embodiment, the invention provides processes for preparing a premix for use in pharmaceutical formulations of dexlansoprazole, an embodiment of a process including: (a) dissolving dexlansoprazole or a pharmaceutically acceptable salt thereof in a solvent; (b) adding a water-soluble excipient to the solution; (c) removing the solvent; (d) treating the residue with an aliphatic hydrocarbon solvent until solids separate; and (e) isolating said solids thereby obtaining a premix.

In embodiments, the process further includes adding a base before the solvent is removed.

In yet another aspect, the invention provides processes for preparing premixes for use in pharmaceutical formulations of dexlansoprazole, an embodiment of a process including: a) suspending dexlansoprazole or a pharmaceutically acceptable salt thereof, a water soluble excipient, and a basic compound in water or an organic solvent; and b) spray-drying the suspension.

The premixes can be used directly, or used in combination with additional excipients, to prepare desired pharmaceutical dosage forms. In other embodiments, the invention includes methods of preparing dosage forms of the present invention.

In further embodiments the invention includes methods of treating patients suffering from gastric-acid related diseases, including, e.g., reflux esophagitis, gastritis, duodenitis, gastric ulcers and duodenal ulcers, using pharmaceutical formulations of the present invention.

Further features of the invention will be apparent from the detailed description herein below set forth.

### BRIEF DESCRIPTION OF THE DRAWINGS

FIG. 1 is an X-ray powder diffraction (XRPD) pattern of amorphous dexlansoprazole.

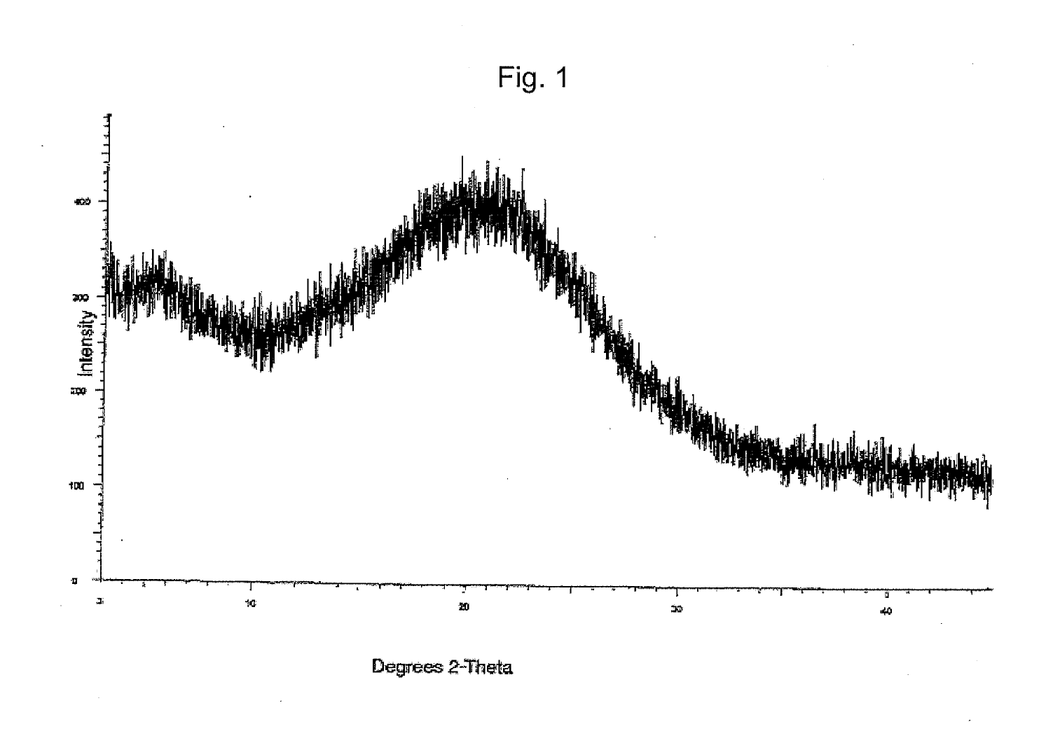

FIG. 2 is an XRPD pattern of a dexlansoprazole premix prepared in Example 1.

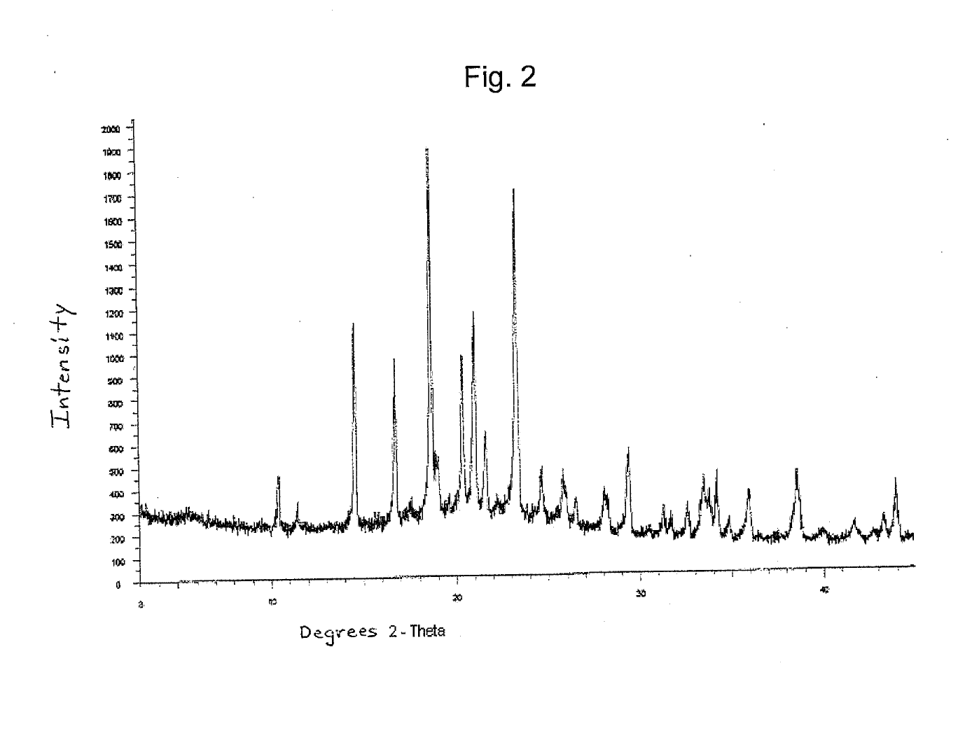

FIG. 3 is an XRPD pattern of a dexlansoprazole premix prepared in Example 2.

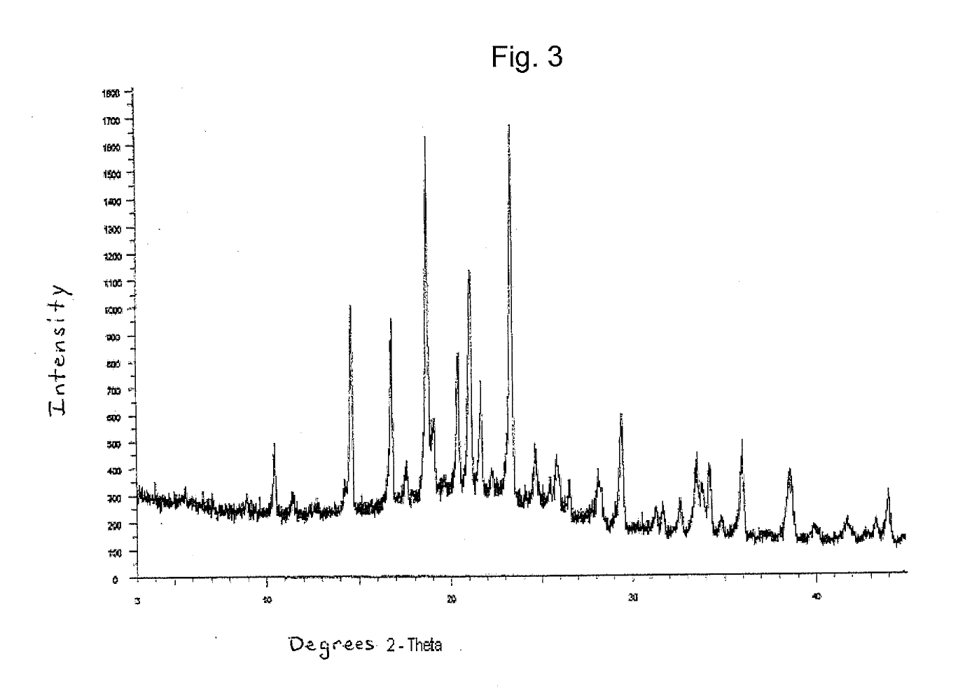

FIG. 4 shows comparative XRPD patterns of mannitol (A), a premix prepared in Example 2 (B), a premix prepared in Example 1 (C), and meglumine (D).

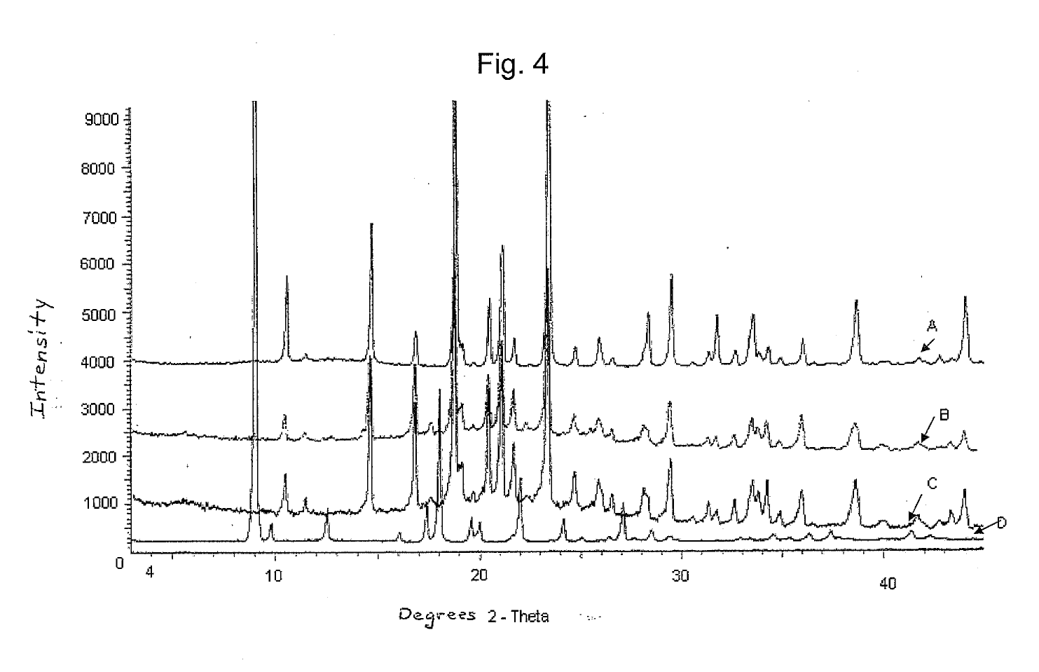

FIG. 5 is an XRPD pattern of a dexlansoprazole premix prepared in Example 6A.

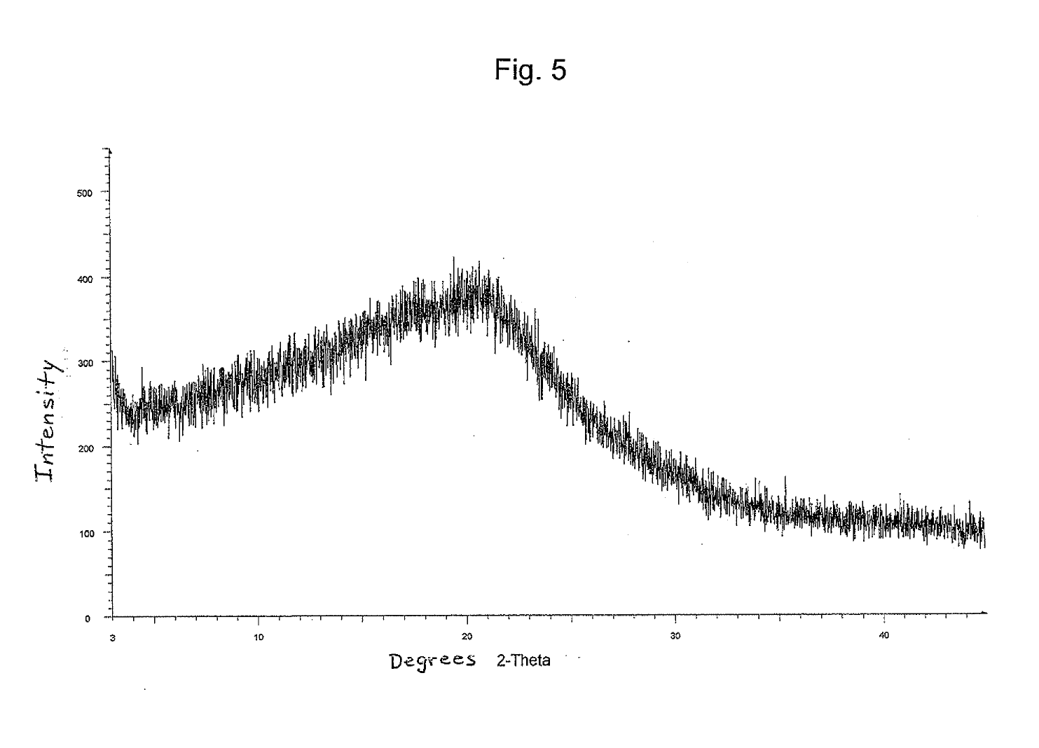

FIG. 6 is an XRPD pattern of a dexlansoprazole premix prepared in Example 6C.

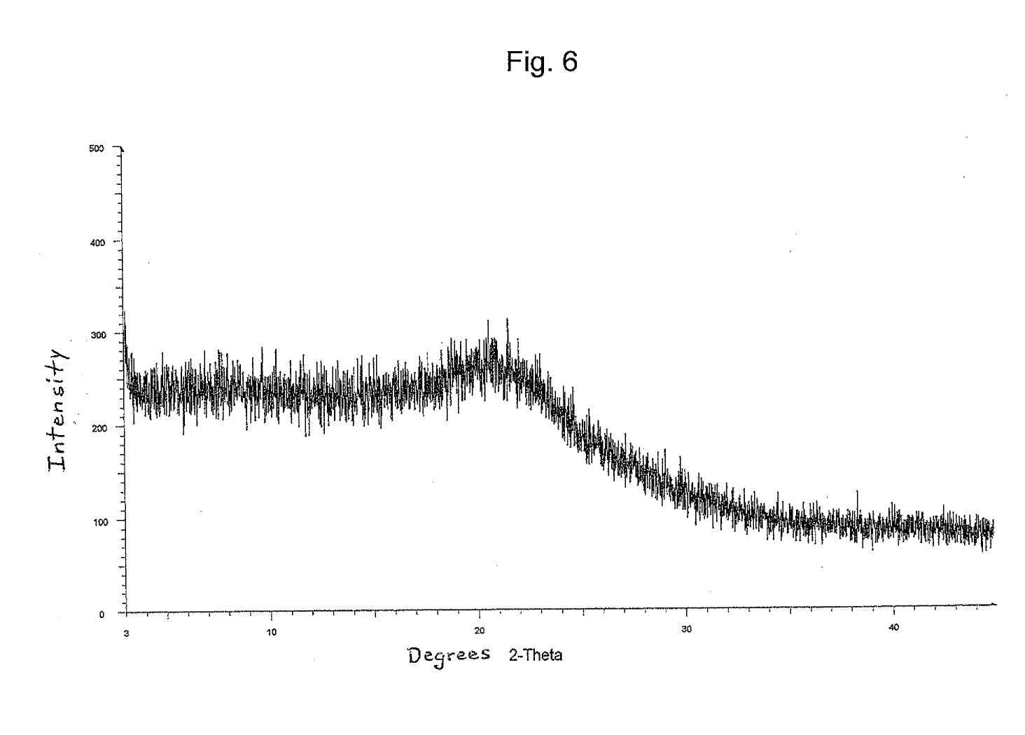

FIG. 7 is an XRPD pattern of a dexlansoprazole premix prepared in Example 6B.

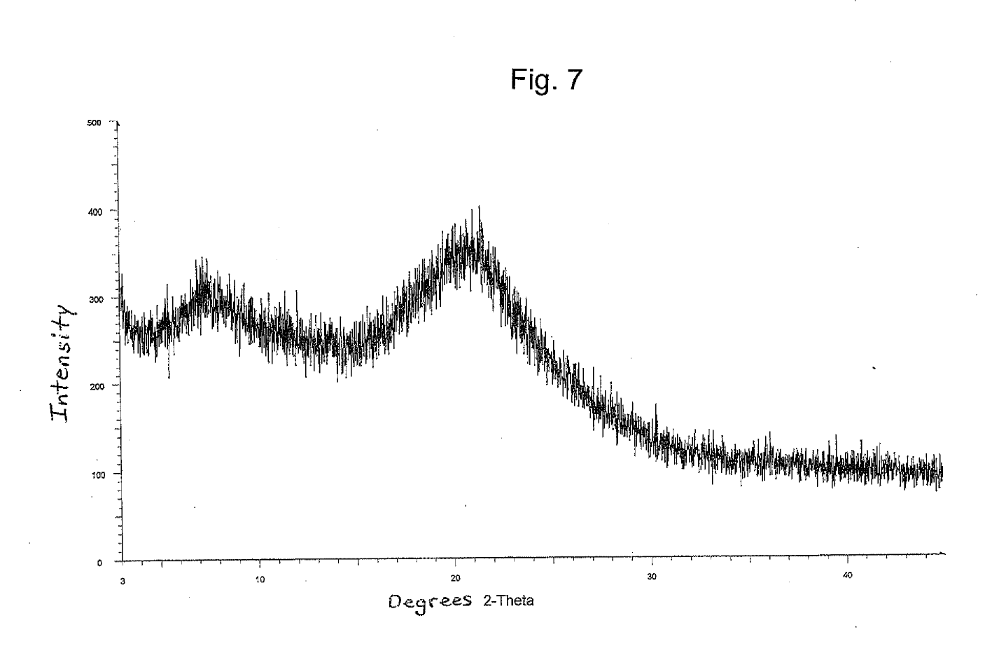

### DETAILED DESCRIPTION

The present invention relates to premix compositions comprising dexlansoprazole, pharmaceutical formulations containing the premixes, and processes for preparing the same.

As used herein the term “dexlansoprazole” includes the compound dexlansoprazole, pharmaceutically acceptable salts thereof, prodrugs thereof, the active metabolites of dexlansoprazole and the prodrugs thereof, and any of their polymorphs, solvates and hydrates.

The terms “pharmaceutically acceptable salt” as used herein refers to salts which are known to be non-toxic and are commonly used in pharmaceutical practice. Such pharmaceutically acceptable salts include metal salts, salts with organic bases, salts with basic amino acids, etc. Metal salts include, for example, alkali metal salts, such as sodium salt and potassium salts, and alkaline earth metal salts, such as calcium, magnesium and barium salts. Salts with organic bases include, for example, salts with trimethylamine, triethylamine, pyridine, picoline, ethanolamine, diethanolamine, triethanolamine, dicyclohexylamine, N,N-dibenzyl ethylenediamine, etc. Salts with basic amino acids include, for example, salts with arginine, lysine, etc.

In the present invention, dexlansoprazole and its salts can be used in any crystalline form, amorphous form, or combinations thereof.

The term “premix” is used herein to describe combinations of dexlansoprazole, including any of its salts, etc., and at least one pharmaceutical excipient, wherein individual particles of the components cannot be distinguished using techniques such as optical microscopy. In embodiments, the drug is considered as being uniformly or non-uniformly distributed over surfaces of excipient particles. In other embodiments, the premixes are considered to be in the nature of molecular dispersions, or solid solutions. Simple mixtures of powdered ingredients will not constitute premixes. Some methods for preparing premixes are described herein.

The term “excipient” means a component of a pharmaceutical product that is not an active ingredient, such as a filler, diluent, carrier, etc. The excipients that are useful in preparing a pharmaceutical composition are generally safe, non-toxic and neither biologically nor otherwise undesirable, and are acceptable for veterinary use as well as human pharmaceutical use. “Pharmaceutically acceptable excipient” as used in the specification and claims includes both one and more than one such excipient.

The term “acid-labile compound” means any compound that is not stable in acidic conditions or which undergoes degradation or hydrolysis via acid or proton catalyzed reactions.

Like other substituted benzimidazole derivatives, dexlansoprazole is acid-labile, creating several problems in formulating into oral pharmaceutical dosage forms because of the acidic environment that will be encountered in the stomach. It has poor stability and would be rapidly decomposed and colored under moist conditions or in an acidic to neutral aqueous environment. It requires special techniques to avoid contact of the drug with gastric acid of the stomach. Even though stabilization of substituted benzimidazole derivatives is known in the art, there remains a need for alternate approaches to prepare stable pharmaceutical compositions comprising dexlansoprazole or a pharmaceutically acceptable salt thereof.

In an embodiment, the invention includes premixes for use in pharmaceutical formulations of dexlansoprazole, said premixes comprising in combination: (a) dexlansoprazole or a pharmaceutically acceptable salt thereof; and (b) a water-soluble excipient.

Useful water soluble excipients include any pharmaceutically acceptable water soluble sugar excipients, preferably having low hydroscopicity, and include, for example, mannitol, lactose, fructose, sorbitol, xylitol, maltodextrin, dextrates, dextrins, lactitol, and mixtures of any two or more thereof. Further, the water soluble excipients according to the present invention include polymers such as, but not limited to, hydroxypropyl celluloses, hydroxypropyl methylcelluloses, polyvinylalcohols, polyvinylpyrrolidones, and mixtures thereof.

In an embodiment, the invention includes premixes for use in pharmaceutical formulations of dexlansoprazole, said premixes comprising in combination: (a) dexlansoprazole or a pharmaceutically acceptable salt thereof; and (b) mannitol.

In an embodiment, the invention includes premixes for use in pharmaceutical formulations of dexlansoprazole, said premixes comprising in combination: (a) dexlansoprazole or a pharmaceutically acceptable salt thereof; and (b) a polyvinylpyrrolidone.

In an embodiment, a premix further comprises a pharmaceutically acceptable basic compound, which serves as a stabilizer for the drug. The stabilizers useful in present invention include, but are not limited to, basic inorganic salts and organic compounds. Various useful basic inorganic salts include but are not limited to basic inorganic salts of sodium, potassium, magnesium and calcium. Examples of basic inorganic salts of sodium are sodium carbonate, sodium hydrogen carbonate, sodium hydroxide, and the like. Examples of basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium metasilicate aluminate, magnesium silicate, magnesium aluminate, synthetic hydrotalcite [Mg₆Al₂(OH)₁₆.CO₃.4H₂O], and aluminum hydroxide-magnesium oxide [2.5MgO.Al₂O₃.xH₂O], and the like. Examples of basic inorganic salts of calcium include precipitated calcium carbonate, calcium hydroxide and the like. Examples of organic bases that may be used in the present invention are pharmaceutically acceptable organic bases, including, without limitation thereto, meglumine, lysine, N,N′-dibenzylethylenediamine, chloroprocaine, choline, diethanolamine, ethylenediamine, procaine, and mixtures of any two or more thereof.

In an embodiment, the invention includes premixes for use in preparing pharmaceutical formulations of dexlansoprazole, said premixes comprising in combination: (a) dexlansoprazole or a pharmaceutically acceptable salt thereof; (b) mannitol; and (c) meglumine.

In an embodiment, the invention includes premixes for use in preparing pharmaceutical formulations of dexlansoprazole, said premixes comprising in combination: (a) dexlansoprazole or a pharmaceutically acceptable salt thereof; (b) mannitol; and (c) magnesium carbonate.

In an embodiment, the invention includes premixes for use in preparing pharmaceutical formulations of dexlansoprazole, said premixes comprising in combination: (a) dexlansoprazole or a pharmaceutically acceptable salt thereof; (b) hydroxypropyl methylcellulose; and (c) meglumine.

In an embodiment, the invention includes premixes for use in preparing pharmaceutical formulations of dexlansoprazole, said premixes comprising in combination: (a) dexlansoprazole or a pharmaceutically acceptable salt thereof; (b) a polyvinylpyrrolidone; and (c) magnesium carbonate.

In embodiments, premixes may be prepared by spray drying a suspension or solution of dexlansoprazole and a water soluble excipient, with or without an organic base. Alternatively, dexlansoprazole premixes may also be prepared using fluid bed granulation techniques, where a solution of dexlansoprazole, with or without basic compound, is sprayed onto a water soluble excipient. In one specific embodiment, a premix may be prepared by a process including: (a) dissolving dexlansoprazole or a pharmaceutically acceptable salt thereof in an organic solvent; (b) combining the solution with a water-soluble sugar derivative; (c) evaporating solvent from the mixture formed in step (b); (d) adding an aliphatic hydrocarbon to the residue formed in step (c); (e) stirring the mixture formed in step (d); and (f) isolating a solid.

After a water soluble sugar derivative is combined with the solution of step (a), an aliphatic hydrocarbon solvent such as cyclohexane, n-heptane, hexane or mixtures thereof may be added. Optionally, the solution of step (a) can be purified with charcoal before combining with a water soluble sugar derivative.

In an embodiment, the invention provides processes for preparing a premix for use in pharmaceutical formulations of dexlansoprazole, an embodiment of a process including: (a) dissolving a water-soluble excipient in a solvent; (b) adding dexlansoprazole or a pharmaceutically acceptable salt thereof to the solution; and (c) spray drying the solution to form a premix.

Solvents that may be used in the present invention include, but are not limited to: halogenated hydrocarbons such as dichloromethane, 1,2-dichloroethane, chloroform and carbon tetrachloride; alcohols such as methanol, ethanol, isopropyl alcohol, 1-propanol, 2-propanol, 1-butanol, 2-butanol, and t-butyl alcohol; ketones such as acetone, ethyl methyl ketone, diethyl ketone, and methyl isobutyl ketone; esters such as ethyl acetate, n-propyl acetate, n-butyl acetate and t-butyl acetate; ethers such as diethyl ether, dimethyl ether, diisopropyl ether, methyl t-butyl ether and 1,4-dioxane; nitriles such as acetonitrile and propionitrile; water; and mixtures thereof.

The aliphatic hydrocarbon of step (d) includes compounds such as, for example, cyclohexane, n-heptane, hexane, and mixtures thereof. The evaporation can be conducted under reduced pressure at low temperatures, such as below about 30° C., or about room temperature, to maintain high purity of the drug compound. Other temperatures are also suitable.

The isolated solids may be dried under reduced pressure at low temperatures, such as about 30-35° C., to obtain a water content below about 2% by weight.

When a premix composition is prepared with an organic base in accordance with one aspect of the present invention, the organic base may be added the solution of step (a) along with a water soluble sugar derivative.

The weight ratio of dexlansoprazole to the pharmaceutically acceptable excipient in a premix is not critical for the invention and may be selected by the skilled practitioner depending on the desired use. The dexlansoprazole premixes typically have weight ratios of dexlansoprazole to the pharmaceutically acceptable excipient from about 2:1 to about 1:10, or from about 1:1 to about 1:6, or from about 1:1 to about 1:4. The pharmaceutically acceptable excipient can be a mixture of more than one compound.

The different physicochemical properties of the active ingredient and as well as of excipients are to be considered, as these properties affect the process and formulation properties. Various important physicochemical properties include but are not limited to particle size, density (bulk density and tapped density), compressibility index, Hausner's ratio, angle of repose, etc. Particle sizes of active pharmaceutical ingredient can affect the solid dosage form in numerous ways. For example, drug content uniformity of pharmaceutical dosage units can be affected by particle sizes and size distribution. This will be even more critical for low-dose drugs and satisfactory dosage units of low doses cannot be manufactured from a drug that does not meet certain particle size and size distribution criteria. Also particle size plays an important role in dissolution of active ingredient form the final dosage form for certain drugs like dexlansoprazole because of their low solubility. Hence, these physicochemical properties not only affect the process of the preparing the pharmaceutical compositions but also affect the performance of the pharmaceutical product both in vitro and in vivo.

The physicochemical properties of the dexlansoprazole premix of the invention can be readily controlled through the choice of appropriate pharmaceutically acceptable excipients that are used in premix preparation. Thus, for example, the particle sizes and distribution of the dexlansoprazole premix of the invention can be readily controlled by the proper choice of the pharmaceutically acceptable excipients with a defined particle size and distribution. Thus, if a larger particle size premix is required, an excipient having the required large particles should be appropriately chosen and, vice versa, if a smaller particle size premix is desired. The selection of appropriate particle sizes of dexlansoprazole as well as of excipients is within the scope of the invention, Mixing of more than one particle size excipient species is also within the scope of the invention. Also, included are mixtures of premixes of dexlansoprazole wherein the excipients that are used in premix preparation, are different.

The D₁₀, D₅₀, and D₉₀ values are useful ways for indicating a particle size distribution. D₉₀ is the size value where at least 90 volume percent of the particles have a size smaller than the said value; Likewise D₁₀ refers to 10 volume percent of the particles having a size smaller than the said value. D₅₀ refers to at least 50 volume percent of the particles having a size smaller than the said value and D_{[4,3]} value refers to the mean particle size. Methods for determining D₁₀, D₅₀ D₉₀ and D_{[4,3]} include laser light diffraction, such as using equipment sold by Malvern Instruments Ltd., Malvern, Worcestershire, United Kingdom. Other types of equipment may be used, as is known in the art.

Flowability of materials is measured and represented using the Carr Index. The Carr Index is the percent ratio of the difference between tapped density and bulk density to tapped density described as:

Carr Index=[(Tapped density−Bulk density)÷Tapped density]×100.

The densities can be determined using the standard test method 616 “Bulk Density and Tapped Density” in United States Pharmacopeia 29, United States Pharmacopeial Convention, Inc., Rockville, Md., 2005.

Carr Index values below about 15% represent materials with very good flow properties and values above about 40% represent materials with very poor flow properties. The dexlansoprazole premixes of the present invention typically have a Carr Index which is substantially lower than the 40% described for products with poor flow properties. Values for Carr Index for the dexlansoprazole premixes of the invention are generally less than about 35%, or less than about 30%, or less than about 25%, or less than about 20%, or less than about 15%. This indicates superior handling capabilities during processing into pharmaceutical dosage forms.

The dexlansoprazole premixes of the invention can be further processed into various pharmaceutical dosage forms as prepared, or can be combined with one or more pharmaceutically acceptable excipients. The different pharmaceutical dosage forms where the dexlansoprazole premixes of the invention find utility include: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release, controlled release or their combinations. Further, immediate release formulations may be conventional, dispersible, chewable, mouth dissolving, or flash melt preparations. Delayed release or controlled release formulations may comprise hydrophilic, lipophilic, or hydrophobic release rate controlling substances, or their combinations to form matrix or reservoir, or combinations of matrix and reservoir systems. The formulations may be prepared using any of direct blending, dry granulation, wet granulation, or extrusion and spheronization. Formulations may be presented as uncoated, film coated, sugar coated, powder coated, enteric coated or controlled release coated forms. Formulations of the present application may further comprise one or more pharmaceutically acceptable excipients.

As used herein the term “controlled release” means that the release of the active substance, i.e., dexlansoprazole, from a pharmaceutical dosage form is controlled in a manner modified to occur at a different time and/or at a different rate than that obtained from an immediate release product, such as a conventional swallowed tablet or capsule. As used herein the terms “delayed release” or “enteric coated” means the release of the active substance is modified to occur at a later time than that from an immediate release form.

In an embodiment, the invention includes oral pharmaceutical formulations in a solid dosage form which include: (a) a core containing a dexlansoprazole premix, which is free of basic substances; (b) a subcoating coated onto the core; and (c) an enteric coating coated onto the subcoating. in certain embodiments, the subcoating is chemically inert.

In an embodiment, the invention includes oral pharmaceutical formulations in a solid dosage form that include: a) a core containing a dexlansoprazole premix, including a base; and b) an enteric coating. In embodiments, the core is substantially free of inorganic basic substances. In an embodiment, an enteric coating is coated directly onto the core. In another embodiment, the oral pharmaceutical formulations further include a subcoating coated onto the core, with the enteric coating applied onto the subcoating.

The cores may also include pharmaceutically acceptable excipients such as surfactants, disintegrants, stabilizers, and/or binders. The cores of the present invention may be prepared by homogenously mixing the premix and pharmaceutically acceptable excipients mentioned hereinabove. The powder mixture is then formulated into small beads, pellets, granules, fine granules, mini-tablets or tablets, hard gelatin or soft gelatin capsules by conventional solid dosage pharmaceutical procedures.

An inert subcoating separates a core from an enteric coating polymer that contains free carboxyl groups, which may cause degradation and/or discoloration. The inert subcoating may also serve as a pH-buffering zone in which hydrogen ions diffusing from the outside toward the alkaline core can react with hydroxyl ions diffusing from the alkaline core toward the surface of the coated articles. A subcoating may be formed by a plural number of layers.

An inert subcoating, or separating layer, can be applied to core pellets or tablets by conventional coating procedures in a suitable coating pan or in fluidized bed apparatus using water and/or an organic solvent for the coating solutions or dispersions. Water soluble or insoluble polymers that can be used for an inert subcoating include, for example, sugars, zein, cellulose derivatives such as hydroxypropyl celluloses, hydroxypropyl methylcelluloses, ethylcelluloses, and hydroxyethyl celluloses, polyvinylalcohols, providones, polyethylene glycols, poloxamers, gelatin, polylysine, polyarginine, polyglycine, polyvinylpyrrolidines, vinyl acetate copolymer, and mixtures thereof.

In the case of tablets, the coatings may also be applied using a dry coating technique. The inert subcoating may also include pharmaceutically acceptable water-soluble or tablet excipients that rapidly dissolve or disintegrate in water. Ordinary plasticizers, pigments, titanium dioxide, talc and other additives may also be included into an inert subcoating. In the case of gelatin capsules, the gelatin capsule itself serves as a subcoating. The quantity of the inert subcoating of the present invention may vary from about 0.3% to 6%, or about 0.5 to 4%, or about 1-3%, of the total weight of a core.

The enteric coating can be applied either directly onto the core or onto the subcoated cores by conventional coating techniques such as, for instance, pan coating or fluidized bed coating using solutions of polymers in water and/or suitable organic solvents, or by using latex suspensions of said polymers. Enteric coating polymers that can be used, for example, include cellulose acetate phthalates (CAP), hydroxypropyl methylcellulose phthalates (HPMCP), polyvinyl acetate phthalates (PVAP), hydroxypropyl methylcellulose acetate succinates (HPMCAS), cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (Gantrez™ ES series), ethyl methyacrylate-methylmethacrylate-chlorotrimethylammonium ethyl acrylate copolymers, natural resins such as zein, shellac and copal collophorium, carboxymethyl ethylcelluloses, co-polymerized methacrylic acid/methacrylic acid methyl esters such as, for instance, materials sold under the trade name EUDRAGIT® L12.5, L100, or EUDRAGIT® S12.5, S100, and several commercially available enteric dispersion systems (e.g., EUDRAGIT® L30D55, EUDRAGIT® FS30D, EUDRAGIT® L100-55, EUDRAGIT® S100 (Evonik Industries, Germany), KOLLICOAT® MAE30D and 30DP (BASF), ESTACRYL® 30D (Eastman Chemical), AQUATERIC® and AQUACOAT® CPD30 (FMC), and mixtures thereof.

The enteric coating layer can optionally contain a pharmaceutically acceptable plasticizer such as, for instance, cetanol, triacetin, citric acid esters such as, for instance, those known under the trade name Citroflex® (Pfizer, New York), phthalic acid esters, dibutyl succinate or similar plasticizers. The amount of plasticizer is usually optimized for each enteric coating polymer and is usually in the range of about 1-20% of the enteric coating polymer. Dispersants such as talc, colorants and pigments may also be included into the enteric coating layer. The weight of enteric coating applied is about 1-12%, or about 2-10%, or about 4-8%, of the weight of core material of the tablet.

In another embodiment, the invention includes oral pharmaceutical compositions in solid dosage forms which include: (a) a core containing a dexlansoprazole premix, which is substantially free of basic substances; and (b) a controlled release coating applied onto the core.

In another embodiment, the invention includes oral pharmaceutical compositions in solid dosage forms that include: a) a core containing a dexlansoprazole premix, including a basic substance; and (b) a controlled release coating applied onto the core.

In an embodiment, a controlled release coating is applied directly onto the core. In another embodiment, the oral pharmaceutical compositions further include a subcoating on the core, with the controlled release coatings applied onto the subcoated core. It is frequently desirable from the viewpoint of improving the stability of dexlansoprazole that the subcoating is provided to prevent direct contact of active ingredient-containing core particles with the release-controlling coating layer.

The controlled release coating is applied either directly onto the core or onto the subcoated cores by conventional coating techniques such as, for instance, pan coating or fluidized bed coating using solutions of polymers in water and/or suitable organic solvents, or by using latex suspensions of said polymers.

In an embodiment, the cores contain one or more release controlling polymers in admixture with dexlansoprazole premix to form a matrix. In certain embodiments, a controlled release matrix is further coated with enteric polymers or controlled release polymers, or combinations thereof.

One or more polymers that can be used in present invention for controlled release include hydrophilic, hydrophobic and lipophilic substances, and combinations thereof. Examples of polymers include, without limitation thereto, cellulose ethers, e.g., hydroxypropyl methylcelluloses (hypromelloses or HPMC), hydroxypropylcelluloses (HPC), hydroxyethylcelluloses, ethylcelluloses, and carboxymethylcellulose sodium, polyvinylpyrrolidones, including non-crosslinked polyvinylpyrrolidones, carboxymethylstarch, polyethylene glycols, polyoxyethylenes, poloxamers (polyoxyethylene-polyoxypropylene copolymers), polyvinylalcohols, glucanes (glucans), carrageenans, scleroglucanes (scleroglucans), mannans, galactomannans, gellans, alginic acid and derivatives (e.g., sodium or calcium alginate, propylene glycol alginate), polyaminoacids (e.g. gelatin), methyl vinyl ether/maleic anhydride copolymers, polysaccharides (e.g. carageenan, guar gum, xanthan gum, tragacanth and ceratonia), alpha-, beta- or gamma-cyclodextrins, dextrin derivatives (e.g. dextrin), polymethacrylates (e.g. copolymers of acrylic and methacrylic acid esters containing quaternary ammonium groups), acrylic acid polymers (e.g., carbomers), shellac and derivatives thereof, cellulose acetate, cellulose butyrate, cellulose diacetate, cellulose triacetate, cellulose propionate, cellulose acetate butyrate and other acetylated cellulose derivatives, etc.

Examples of lipophilic substances that can be used in the present invention include, without limitation thereto, waxes (e.g., carnauba wax, microcrystalline wax, beeswax, and polyethoxylated beeswax), natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, etc.

In an embodiment, the invention includes controlled release pharmaceutical formulations comprising dexlansoprazole premix, wherein said compositions are in multiparticulate form.

In another embodiment, the invention includes controlled release pharmaceutical formulations comprising cores comprising dexlansoprazole premix and a coating comprising one or more controlled release polymers, enteric polymers or combinations thereof, and said formulations are in multiparticulate form.

In an embodiment, the dexlansoprazole premix formulations of the present invention comprise a single fraction of multiparticulates, such as pellets or minitablets, filled into a capsule wherein the multiparticulate fraction comprises cores containing the active agent for providing extended release, optionally having a coating layer containing the active agent which at least partially covers the core and provides immediate release of the active agent, and which are further coated with an enteric polymer, and wherein the multiparticulates are optionally coated to form a subcoating layer prior to the enteric coating.

In another embodiment, the invention provides modified release formulations comprising dexlansoprazole premix which comprise at least two fractions of multiparticulates wherein one or more of said fractions provide immediate release, delayed release, extended release, sustained release, pulsatile release, or prolonged release of the active agent.

In an aspect, modified release formulations of dexlansoprazole premixes according to the present invention comprise at least two fractions wherein both of the fractions provide modified release of dexlansoprazole. In an embodiment, the modified release formulations of dexlansoprazole premix comprise at least two fractions wherein both the fractions provide delayed release of dexlansoprazole, following administration, such that the drug release of one delayed release fraction precedes the other delayed release fraction while releasing a substantial amount of drug before, at the same time, or after a substantial amount of drug is released from the other fraction. In another embodiment, the modified release formulations of dexlansoprazole premix comprise at least two fractions wherein both fractions are in the form of enteric coated compositions intended to provide delayed release of dexlansoprazole, and wherein at least one of the delayed release fractions provides the drug release almost immediately or in an extended manner. Another embodiment comprises a delayed release fraction and a fraction that provides an extended release profile of the drug, the onset of release beginning at a time that is delayed after administration.

In an embodiment, controlled release multiparticulates comprising a dexlansoprazole premix comprise non-pariel cores such as inert sugar or similar substances, upon which dexlansoprazole premix is coated, optionally together with pharmaceutically acceptable excipients, using any technique such as powder layering, solution spraying, or suspension spraying.

In an embodiment, controlled release formulations of the invention comprise dexlansoprazole premix-loaded non-pariel cores having a coating comprising one or more controlled release polymers, enteric polymers or combinations thereof.

In an embodiment, the invention includes pharmaceutical formulations comprising controlled release multiparticulates comprising dexlansoprazole premix, comprising premix-containing cores, and a coating comprising one or more polymers, and optionally having one or more further coatings.

In still other embodiments, multiparticulates comprising dexlansoprazole premix further contain a non-functional seal coating, a functional coating, or both.

In further embodiments, any one or all of the coating compositions optionally contain dexlansoprazole premix.

The multiparticulate formulations of the invention can be prepared using the techniques described herein, as well as other methods known to those having skill in the art.

In an embodiment, multiparticulates comprising dexlansoprazole premix are coated with different concentrations of polymers, giving portions having different release profiles, and these can be combined to form a pharmaceutical formulation or dosage form to achieve desired controlled release profiles.

In another embodiment, multiparticulates comprising dexlansoprazole pre-mix are coated with different types of polymers, either enteric polymers or controlled release polymers, giving different release profiles, and these can be combined to form a pharmaceutical formulation or dosage form to achieve desired controlled release profiles.

In another embodiment, multiparticulates comprising dexlansoprazole pre-mix can be combined with pharmaceutically acceptable excipients, and compounded to form a pharmaceutical formulation, i.e., can be compressed into tablets or placed into suitable capsule shells, using techniques known to those having skill in the art.

Pharmaceutically acceptable excipients may be utilized as required for conversion of the premixes into the final pharmaceutical dosage forms and include, for example, any one or more of diluents, binders, stabilizers, lubricants, glidants, disintegrating agents, surfactants, and other additives that are commonly used in solid pharmaceutical dosage form preparations.

### Diluents:

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different starches include but are not limited to maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH101, PH102, PH301, PH302 and PH-F20, PH112 microcrystalline cellulose 114, and microcrystalline cellulose 112. Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include but are not limited to hydroxypropylcelluloses, also called HPC (Klucel™ LF, Klucel EXF) and useful in various grades, hydroxypropyl methylcelluloses, also called hypromelloses or HPMC (Methocel™) and useful in various grades, polyvinylpyrrolidones or povidones (such as grades PVP-K25, PVP-K₂₉, PVP-K30, and PVP-K90), Plasdone™ S-630 (copovidone), powdered acacia, gelatin, guar gum, carbomers (Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Stabilizers:

Various useful stabilizers include basic inorganic salts, such as but not limited to basic inorganic salts of sodium, potassium, magnesium and calcium. Examples of basic inorganic salts of sodium are sodium carbonate, sodium hydrogen carbonate, sodium hydroxide, and the like. Examples of basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium metasilicate aluminate, magnesium silicate, magnesium aluminate, synthetic hydrotalcite [Mg₆Al₂(OH)₁₆.CO₃.4H₂O], aluminum hydroxide-magnesium [2.5MgO.Al₂O₃.xH₂O], and the like. Examples of basic inorganic salts of calcium include precipitated calcium carbonate, calcium hydroxide, and the like.

### Surface-Active Agents:

Useful surface-active agents include non-ionic, cationic and anionic surface-active agents. Useful non-ionic surface-active agents include ethylene glycol stearates, propylene glycol stearates, diethylene glycol stearates, glycerol stearates, sorbitan esters (SPAN™) and polyhydroxyethylenically treated sorbitan esters (TWEEN™), aliphatic alcohols and PEG ethers, phenol and PEG ethers. Useful cationic surface-active agents include quaternary ammonium salts (e.g. cetyltrimethylammonium bromide) and amine salts (e.g. octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk.

### Lubricants:

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

Glidants:

One or more glidant materials, which improve the flow of powder blends and minimize dosage form weight variations can be used. Useful glidants include but are not limited to silicone dioxide, talc and combinations thereof.

### Colouring Agents:

Colouring agents can be used to colour code the compositions, for example, to indicate the type and dosage of the therapeutic agent therein. Suitable colouring agents include, without limitation, natural and/or artificial compounds such as FD&C colouring agents, natural juice concentrates, pigments such as titanium oxide, silicon dioxide, iron oxides, zinc oxide, combinations thereof, and the like.

### Solvents:

Various solvents can be used in the processes of preparation of pharmaceutical compositions and dosage forms of the present invention, including but not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran, and mixtures thereof.

Useful additives for coatings include but are not limited to plasticizers, antiadherents, opacifiers, solvents, and optionally colorants, lubricants, pigments, antifoam agents, and polishing agents.

Various useful plasticizers include but are not limited to substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titianium dioxide may also be present, typically in an amount ranging from about 10% to about 20% based on the total weight of the coating.

Antiadhesives are frequently used in the film coating process to avoid sticking effects during film formation and drying. An example of a useful antiadhesive for this purpose is talc. The antiadhesive is frequently present in the film coating in an amount of about 5% (w/w) to 15% (w/w) based upon the total weight of the coating.

When coloured tablets are desired, a colour is normally applied in the coating. Consequently, colouring agents and pigments may be present in the film coating. Various colouring agents include but are not limited to iron oxides, which can be red, yellow, black or blends thereof.

Suitable polishing agents include polyethylene glycols of differing molecular weights and mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In some embodiments, polyethylene glycols having molecular weights of 3,000-20,000 are employed.

In addition to above coating ingredients, sometimes pre-formulated coating products such as those sold as OPADRY™ (supplied by Colorcon) can conveniently be used. Opadry compositions generally comprise polymer, plasticizer and, if desired, pigment in a dry concentrate that requires only dispersion in a liquid prior to use. Opadry formulas produce attractive, elegant coatings on a variety of tablet cores and can be used in both aqueous and organic coating procedures.

The foregoing descriptions of excipients are not intended to be exhaustive. Those skilled in the art will be aware of many other substances that are useful in the practice of the invention, and the use of such substances is specifically included in this invention.

In embodiments, the invention includes methods of preparing the pharmaceutical compositions of the present invention.

Equipment suitable for processing the pharmaceutical compositions of the present invention include any one or more of rapid mixer granulators, planetary mixers, mass mixers, ribbon mixers, fluid bed processors, mechanical sifters, blenders, roller compactors, extrusion-spheronizers, compression machines, capsule filling machines, rotating bowls or coating pans, tray dryers, fluid bed dryers, rotary cone vacuum dryers, and the like, multimills, fluid energy mills, ball mills, colloid mills, roller mills, hammer mills, and the like, equipped with a suitable screen.

In an aspect, the invention also provides methods of treating gastrointestinal inflammatory diseases and gastric acid-related diseases in mammals and man including reflux esophagitis, gastritis, duodenitis, gastric ulcer and duodenal ulcer, using the formulations and pharmaceutical compositions of the present invention. The compounds and compositions of this invention may be administered to a subject in a therapeutically effective amount.

The pharmaceutical dosage forms of the present invention are intended for oral administration to a patient in need thereof.

X-ray diffraction patterns reported herein were obtained using copper Kα radiation.

Certain specific aspects and embodiments of the invention will be described in more detail with reference to the following examples, being provided only for purposes of illustration, and it is to be understood that the present invention is not deemed to be limited thereto.

### Example 1

### Dexlansoprazole Premix with Mannitol

Amorphous dexlansoprazole (5 g) is suspended in acetone (25 mL) and stirred well to form a clear solution. Charcoal (0.5 g) is added and stirred for 15-30 minutes. The mass is filtered through a Hyflow (flux-calcined diatomaceous earth) bed and washed with acetone (15 mL). To the filtrate, mannitol (5 g) and cyclohexane (60 mL) are added, and then the solvent is distilled under reduced pressure at 20-30° C. Cyclohexane (50 mL) is added to the residue and distilled under reduced pressure at 20-30° C. Then cyclohexane (30 mL) is added and the mass is stirred for 15-30 minutes. Solid is then filtered from the mass and dissolved in dichloromethane (200 mL), and the solvent is distilled under reduced pressure at 35-50° C. to obtain the final premix.

### Example 2

### Dexlansoprazole Premix with Mannitol and Meglumine

Amorphous dexlansoprazole (5 g) is suspended in acetone (25 mL) and stirred well to form a clear solution. Charcoal (0.5 g) is added and stirred for 15-30 minutes. The mass is filtered through a Hyflow bed and washed with acetone (15 mL). To the filtrate, meglumine (0.3 g), mannitol (4.3 g) and cyclohexane (60 mL) are added, and then the solvent is distilled under reduced pressure at 20-30° C. Cyclohexane (50 mL) is then added to the residue and distilled under reduced pressure at 20-30° C. Then cyclohexane (30 mL) is added and the mass is stirred for 15-30 minutes. Solid is then filtered from the mass and dissolved in dichloromethane (200 mL), and the solvent is distilled under reduced pressure at 35-50° C. to obtain the final premix.

### Example 3

### Particle Size Distribution Parameters

The premixes of Example 1 and Example 2 are analyzed for particle size distribution using a Malvern instrument and the results are below:

| Material | D₁₀ (μm) | D₅₀ (μm) | D₉₀ (μm) |
|---|---|---|---|
| Amorphous dexlansoprazole | 9.061 | 22.182 | 42.598 |
| Premix of Example 1 | 3.022 | 54.998 | 136.638 |
| Premix of Example 2 | 3.535 | 57.970 | 138.372 |

### Example 4

### Dexlansoprazole Premix with Mannitol

Amorphous dexlansoprazole (5 g) is suspended in dichloromethane (200 mL) and stirred well to form a clear solution. Charcoal (0.5 g) is added and stirred for 15-30 minutes. The mass is filtered through a Hyflow bed and washed with dichloromethane (15 mL). To the filtrate, mannitol (5 g) and cyclohexane (60 mL) are added, and then the solvent is distilled under reduced pressure at 20-30° C. Cyclohexane (50 mL) is then added to the residue and distilled under reduced pressure at 20-30° C. Cyclohexane (30 mL) is added and the mass is stirred for 15-30 minutes. Solid is then filtered from the mass and dried to obtain the final premix.

### Example 5

### Dexlansoprazole Premix with Mannitol and Meglumine

Amorphous dexlansoprazole (5 g) is suspended in dichloromethane (200 mL) and stirred well to form a clear solution. Charcoal (0.5 g) is added and stirred for 15-30 minutes. The mass is filtered through a Hyflow bed and washed with dichloromethane (15 mL). To the filtrate, meglumine (0.3 g), mannitol (4.3 g) and cyclohexane (60 mL) are added, and then the solvent is distilled under reduced pressure at 20-30° C. Cyclohexane (50 mL) is then added to the residue and distilled under reduced pressure at 20-30° C. Cyclohexane (30 mL) is added and the mass is stirred for 15-30 minutes. Solid is then filtered from the mass and dried to obtain the final premix.

### Example 6

### Dexlansoprazole Premixes

| | Grams | | |
|---|---|---|---|
| Ingredient | 6A | 6B | 6C |
| Dexlansoprazole amorphous | 6 | 6 | 6 |
| Polyvinylpyrrolidone (PVP K-30) | 6 | — | — |
| Hydroxypropylmethylcelluose | — | 6 | — |
| (HPMC) 5 cps | | | |
| Polyvinylpyrrolidone (PVP K-90) | — | — | 6 |

Manufacturing Process:

1) PVP or HPMC is dissolved in methanol, then dexlansoprazole is added and dissolved.

2) The solution is spray dried to produce a premix.

### Example 7

### Dexlansoprazole Premix

| Ingredient | Grams |
|---|---|
| Dexlansoprazole amorphous | 6 |
| Hydroxypropylmethylcelluose 5 cps | 6 |
| Magnesium carbonate heavy | 2 |

Manufacturing Process:

1) Hydroxypropyl methylcelluose 5 cps is dissolved in methanol and magnesium carbonate is dispersed in the solution.

2) Dexlansoprazole is dissolved in the dispersion.

3) The dispersion is spray dried to produce a premix.

### Example 8

### Dexlansoprazole Premix

| Ingredient | Grams |
|---|---|
| Dexlansoprazole amorphous | 6 |
| Polyvinylpyrrolidone PVP K-30 | 6 |
| Magnesium carbonate heavy | 2 |

Manufacturing Process:

1) PVP K-30 is dissolved in methanol and magnesium carbonate heavy is dispersed in the solution.

2) Dexlansoprazole is dissolved in the dispersion.

3) The dispersion is spray dried to produce a premix.

### Example 9

### Dexlansoprazole Premix

| Ingredient | Grams |
|---|---|
| Dexlansoprazole amorphous | 6 |
| Hydroxypropylmethylcelluose 5 cps | 6 |
| Meglumine | 0.6 |

Manufacturing Process:

1) Hydroxypropylmethylcelluose 5 cps is dissolved in methanol and meglumine is dissolved in the solution.

2) Dexlansoprazole is dissolved in the solution.

3) The solution is spray dried to produce a premix.

### Example 10

### Premix Glass Transition Temperatures

The glass transition temperature (Tg) is determined for premixes using differential scanning calorimetry and the results are below:

| Material | Tg |
|---|---|
| Dexlansoprazole (amorphous) | 55.48° C. |
| Example 6A | 97.60° C. |
| Example 6B | 70.70° C. |
| Example 6C | 102.61° C. |

The glass transition temperatures of the premix compositions are greater than that of dexlansoprazole (amorphous). This indicates that premixes are more physically stable, as compared to dexlansoprazole (amorphous).

### Example 11

### Dexlansoprazole 30 mg Tablets

| | |
|---|---|
| Ingredient | mg/Tablet |

Core

| | |
|---|---|
| Dexlansoprazole premix (Example 1)* | 60 |
| Magnesium oxide | 20 |
| Mannitol (Pearlitol ™ SD 200) | 158.3 |
| Crosspovidone | 22 |
| Copovidone (Plasdone ™ S-630) | 25 |
| Sodium lauryl sulphate | 3.5 |
| Glycine | 17 |
| Sodium stearyl fumarate | 10 |
| Talc | 3 |
| Colloidal silicon dioxide | 1 |
| Iron oxide red | 0.2 |
| Core Weight | 320 |

Subcoating

| | |
|---|---|
| Zein F6000 | 5.1 |

Enteric Coating

| | |
|---|---|
| Eudragit ® L100-55 (Methacrylic acid copolymer type C) | 17.8373 |
| Triethyl citrate | 1.7837 |
| Talc | 0.333 |
| Titanium dioxide | 0.4459 |

Film Coating

| | |
|---|---|
| Opadry ™ Pink OY** | 7.48 |
| Total Weight | 353 |

*Content of premix: dexlansoprazole 30 mg and mannitol 30 mg.
**Opadry ™ Pink OY is a pre-formulated coating product containing hypromellose, titanium dioxide (E171), macrogol 400 and erythrosine lake (E127), sold by Colorcon.

Manufacturing Process:

1) Mix dexlansoprazole premix with remaining core ingredients.

2) Compress the blend of 1) into tablets.

3) Coat the tablet of 2) with a solution of zein in 90% isopropyl alcohol and 10% water, and dry.

4) Coat the subcoated tablets of 3) with enteric coating ingredients dispersed in isopropyl alcohol, and dry.

5) Coat the enteric coated tablets of 4) with Opadry Pink dispersion in water, and dry.

### Example 12

### Dexlansoprazole 30 mg Tablets

| | |
|---|---|
| Ingredient | mg/Tablet |

Core

| | |
|---|---|
| Dexlansoprazole premix (Example 2)* | 57.6 |
| Magnesium oxide | 20 |
| Mannitol (Pearlitol SD 200) | 161.2 |
| Crospovidone | 22 |
| Copovidone (Plasdone S-630) | 25 |
| Sodium lauryl sulphate | 3.5 |
| Glycine | 17 |
| Sodium stearyl fumarate | 10 |
| Talc | 3 |
| Colloidal silicon dioxide | 1 |
| Core Weight | 320 |

Subcoating

| | |
|---|---|
| Hydroxypropyl methylcellulose (HPMC) 5 Cps | 13.6 |
| Triethyl citrate | 1.4 |

Enteric Coating

| | |
|---|---|
| Eudragit L100-55 | 17.8373 |
| Triethyl citrate | 1.7837 |
| Talc | 0.333 |
| Titanium dioxide | 0.4459 |

Film Coating

| | |
|---|---|
| Opadry ™ Pink OY | 7.48 |
| Total Weight | 363 |

*Content of premix: dexlansoprazole 30 mg, mannitol 25.8 mg, and meglumine 1.8 mg.

Manufacturing Process:

1) Mix dexlansoprazole premix with remaining core ingredients.

2) Compress the blend of 1) into tablets.

3) Coat the tablets of 2) with a solution of HPMC and triethyl citrate in 90% isopropyl alcohol and 10% water, and dry.

4) Coat the subcoated tablets of 3) with enteric coating ingredients dispersed in isopropyl alcohol, and dry.

5) Coat the enteric coated tablets of 4) with Opadry Pink dispersion in water, and dry.

### Example 13

### Dexlansoprazole 30 mg Capsules

| Ingredient | mg/Capsule |
|---|---|
| Sucrose/starch spheres* | 70 |

Core Coating

| | |
|---|---|
| Dexlansoprazole premix (Example 2)** | 57.6 |
| Magnesium carbonate | 14 |
| Sucrose (pulverized) | 27.4 |
| Corn starch | 9 |
| Low substituted hydroxypropyl cellulose | 10 |
| Titanium dioxide | 1 |

Intermediate Coating

| | |
|---|---|
| Sucrose (pulverized) | 5 |
| Corn starch | 2.5 |
| Low-substituted hydroxypropyl cellulose | 2.5 |

Binder

| | |
|---|---|
| Hydroxylpropyl cellulose | 1 |
| Water*** | 49 |

Enteric Coating

| | |
|---|---|
| Methacrylic acid copolymer | 26 |
| Talc | 7.8 |
| Polyethylene glycol | 2.5 |
| Titanium dioxide | 2.5 |
| Polysorbate 80 | 1 |
| Water*** | 119.5 |

Glidant

| | |
|---|---|
| Talc | 0.1 |
| Colloidal silicon dioxide | 0.1 |
| Total Weight | 240 |

*Trade name: Nonpareil-101, supplied by Freund Industrial Co., Ltd., Tokyo, Japan.
**Content of premix: dexlansoprazole 30 mg, mannitol 25.8 mg and meglumine 1.8 mg.
***Evaporates during processing.

Manufacturing Process:

1. Mix dexlansoprazole premix, magnesium carbonate, sucrose, corn starch and low-substituted hydroxypropyl cellulose thoroughly to obtain a dusting powder of active ingredient.

2. Mix sucrose, corn starch and low-substituted hydroxypropyl cellulose thoroughly to obtain a dusting powder for an intermediate layer.

3. Prepare binder solution by dissolving hydroxypropyl cellulose in water to form a 2% w/w solution.

4. Place sucrose/starch spheres in a centrifugal fluid-bed granulator and coat the spheres with the dusting powder of active ingredient of step 1) and the dusting powder for intermediate layer of 2) sequentially on the sucrose/starch spheres while spraying binder solution of 3) to obtain spherical granules.

5. Dry the spherical granules of 4) at 40° C. for 20 hours under vacuum, and sift through a sieve.

6. Prepare the enteric coating dispersion and coat the dried granules of 5), using a fluidized granulation coater.

7. Dry the enteric coated granules of 6) and sift through a sieve.

8. Mix the granules of 7) with talc and colloidal silicon dioxide and fill into a size 3 hard gelatin capsule.

### Example 14

### Dexlansoprazole 30 mg Capsules

| Ingredient | mg/Capsule |
|---|---|
| Sucrose/starch spheres* | 70 |

Core Coating

| | |
|---|---|
| Dexlansoprazole premix (Example 2)** | 57.6 |
| Magnesium carbonate | 14 |
| Sucrose (pulverized) | 27.4 |
| Corn starch | 9 |
| Low substituted hydroxypropyl cellulose | 10 |
| Titanium dioxide | 1 |

Intermediate Coating

| | |
|---|---|
| Sucrose (pulverized) | 5 |
| Corn starch | 2.5 |
| Low-substituted hydroxypropyl cellulose | 2.5 |

Binder

| | |
|---|---|
| Hydroxypropyl cellulose | 1 |
| Water*** | 49 |

Enteric Coating

| | |
|---|---|
| Methacrylic acid copolymer S | 15 |
| Methacrylic acid copolymer L | 13 |
| Talc | 14 |
| Triethyl citrate | 2.8 |
| Water*** | 40.32 |
| Ethanol*** | 362.88 |

Glidant

| | |
|---|---|
| Talc | 0.1 |
| Colloidal silicon dioxide | 0.1 |
| Total Weight | 245 |

*Trade name: Nonpareil-101, supplied by Freund Industrial Co., Ltd., Tokyo, Japan.
**Content of premix: dexlansoprazole 30 mg, mannitol 25.8 mg and meglumine 1.8 mg.
***Evaporates during processing.

Manufacturing Process:

1. Mix dexlansoprazole premix, magnesium carbonate, sucrose, corn starch and low-substituted hydroxypropyl cellulose thoroughly to obtain a dusting powder of active ingredient.

2. Mix sucrose, corn starch and low-substituted hydroxypropyl cellulose thoroughly to obtain a dusting powder for an intermediate layer.

3. Prepare binder solution by dissolving hydroxypropyl cellulose in water to form a 2% w/w solution.

4. Place sucrose/starch spheres in a centrifugal fluid-bed granulator and coat the spheres with the dusting powder of active ingredient of 1) and the dusting powder for intermediate layer of 2) sequentially on the sucrose/starch spheres while spraying binder solution of 3) to obtain spherical granules.

5. Dry the spherical granules of 4) at 40° C. for 20 hours under vacuum and sift through a sieve.

6. Dissolve methacrylic acid copolymer S, methacrylic acid copolymer L and triethyl citrate in a mixed solution of water and ethanol, and disperse talc into the solution to obtain an enteric coating dispersion.

7. Coat the dried granules of 5) with an enteric coating dispersion of 6) using a fluidized granulation coater.

8. Dry the enteric coated granules of 7) and sift through a sieve.

9. Mix the granules of 8) with talc and colloidal silicon dioxide and fill into a size 3 hard gelatin capsule.

### Example 15

### Pharmaceutical Formulation Comprising Two Different Minitablets Filled into Capsules

| | | |
|---|---|---|
| | mg/Capsule | mg/Capsule |
| | (60 mg | (30 mg |
| Ingredient | Drug) | Drug) |

Core

| | | |
|---|---|---|
| Dexlansoprazole premix (Example 8)* | 140 | 60 |
| Magnesium carbonate (heavy) | 28 | 14 |
| Low substituted hydroxypropyl cellulose (L- | 10 | 5 |
| HPC LH31) | | |
| Mannitol (Pearlitol SD200) | 136 | 43 |
| Low substituted hydroxypropyl cellulose (L- | 6 | 3 |
| HPC 11) | | |
| Talc | 15 | 7.5 |
| Sodium stearyl fumarate | 15 | 7.5 |

Subcoating

| | | |
|---|---|---|
| Hydroxypropyl methylcellulose (HPMC) | 9 | 4.5 |
| 5 cps | | |
| Talc | 3.6 | 1.8 |
| Titanium dioxide | 4.8 | 2.4 |
| Isopropyl alcohol** | q.s. | q.s. |
| Methylene chloride** | q.s. | q.s. |

Delayed Release Coating

| | | |
|---|---|---|
| Methacrylic acid copolymer type C (Eudragit | 9.57 | 4.95 |
| L30D-55) | | |
| Polyethylene glycol 6000 | 0.96 | 0.495 |
| Talc | 2.9 | 1.49 |
| Titanium dioxide | 0.96 | 0.495 |
| Polysorbate 80 | 0.11 | 0.06 |
| Water** | q.s. | q.s. |

Extended Release Coating

| | | |
|---|---|---|
| Eudragit S 100 | 58 | 29 |
| Eudragit L 100 | 10.63 | 5.315 |
| Talc | 32.85 | 16.425 |
| Triethyl citrate | 8.02 | 4.01 |
| Isopropyl alcohol** | q.s. | q.s. |
| Water** | q.s. | q.s. |

*Premix composition: dexlansoprazole + PVP K-30 + magnesium carbonate in a 3:3:1 weight ratio.
**Evaporates during processing.

Manufacturing Process:

1. Core

1.1. Mix drug premix, magnesium carbonate, L HPC-31, L HPC-11 and mannitol in a double cone blender for 20 minutes.

1.2. Sift talc and sodium stearyl fumarate through an ASTM #40 mesh sieve.

1.3. Blend the mixtures of 1.1 and 1.2 for 10 minutes.

1.4. Compress the lubricated blend of 1.3 into minitablets having an average weight of 5 mg, using 2 mm round punches.

2. Subcoating 2.1. Dissolve hydroxypropyl methylcellulose in a mixture of isopropyl alcohol and methylene chloride,

2.2. Sift talc and titanium dioxide through an ASTM #60 mesh sieve.

2.3. Disperse talc and titanium dioxide in a mixture of isopropyl alcohol and methylene chloride and circulate through a colloid mill.

2.4. Add dispersion of 2.3 to polymer solution of 2.1 and stir.

2.5. Coat the minitablets of 1.4 with dispersion of 2.4 using a fluid bed processor, to produce a 5% weight gain, after drying.

3. Delayed Release Coating

3.1. Disperse methacrylic acid copolymer type C in water to form a 30% by weight dispersion.

3.2. Dissolve PEG 6000 in water and add dispersion of 3.1. Disperse talc and titanium dioxide in the solution and homogenize for 15 minutes.

3.3. Dissolve polysorbate 80 in warm water and cool.

3.4. Add dispersion of step 3.3 to the solution of 3.2 and stir.

3.5. Spray the dispersion of 3.4 onto subcoated minitablets of 2.5 to produce a weight gain of 15% w/w, after drying, using a fluidized bed processor (FBP).

3.6. Dry enteric coated minitablets in the FBP until loss on drying (LOD) of the pellets is 1-3% w/w at 60° C.

3.7. Cure coated minitablets in the FBP for at 40° C. for 2 hours.

4. Extended Release Coating

4.1. Dissolve Eudragit S100 and Eudragit L100 in a mixture of isopropyl alcohol and water, then dissolve tritethyl citrate in the solution.

4.2. Add talc to the solution with continuous stirring.

4.3. Spray the dispersion of 4.2 onto subcoated minitablets of 2.5 to produce a weight gain of 40% w/w, after drying, using a FBP.

4.4. Dry coated minitablets in the FBP until LOD is 1-3% w/w at 60° C.

4.7. Cure the minitablets in the FBP at 40° C. for 2 hours.

5. Encapsulation

5.1. Fill delayed release minitablets containing 25% of the dexlansoprazole dose, and extended release minitablets containing 75% of the dexlansoprazole dose, into an empty hard gelatin capsule.

### Example 16

### Pharmaceutical Formulation Comprising Two Different Pellets Filled into Capsules

| | | |
|---|---|---|
| | mg/Capsule | mg/Capsule |
| | (60 mg | (30 mg |
| Ingredient | Drug) | Drug) |

Core Pellets
A. Inert Core

| | | |
|---|---|---|
| Sugar spheres (#25/#30 mesh) | 80 | 40 |

B. Drug Powder

| | | |
|---|---|---|
| Dexlansoprazole premix (Example 6A)* | 120 | 60 |
| Magnesium carbonate (heavy) | 48 | 24 |
| Low substituted hydroxypropyl cellulose | 10 | 5 |
| (L-HPC LH31) | | |
| Sucrose (milled) | 10 | 5 |

C. Binder

| | | |
|---|---|---|
| Hydroxypropyl cellulose (Klucel LF) | 2 | 1 |
| Isopropyl alcohol** | q.s. | q.s. |

Subcoating

| | | |
|---|---|---|
| Hydroxypropyl cellulose (Klucel LF) | 15 | 7.5 |
| Talc | 6 | 3 |
| Titanium dioxide | 9 | 4.5 |
| Isopropyl alcohol** | q.s. | q.s. |
| Methylene chloride** | q.s. | q.s. |

Delayed Release Coating

| | | |
|---|---|---|
| Methacrylic acid copolymer type C (Eudragit | 9.9 | 4.95 |
| L30D55) | | |
| Polyethylene glycol 6000 | 0.99 | 0.495 |
| Talc | 2.97 | 1.49 |
| Titanium dioxide | 0.99 | 0.495 |
| Polysorbate 80 | 0.12 | 0.06 |
| Water** | q.s. | q.s. |
| Talc | 1 | 0.5 |

Extended Release Coating

| | | |
|---|---|---|
| Eudragit S 100 | 48 | 24 |
| Eudragit L 100 | 8.4 | 4.2 |
| Talc | 27 | 13.5 |
| Triethyl citrate | 6.6 | 3.3 |
| Isopropyl alcohol** | q.s. | q.s. |
| Water** | q.s. | q.s. |
| Talc | 1 | 0.5 |

*Premix composition: dexlansoprazole + PVP K-30 in a 1:1 weight ratio.
**Evaporates during processing.

Manufacturing Process:

1. Core Pellets

1.1. Mix drug premix, powdered sucrose, magnesium carbonate and L-HPC to form a drug layering powder.

1.2. Dissolve HPC in isopropyl alcohol.

1.3. Use HPC binder solution from 1.2 and drug layering powder from 1.1 to coat the sugar spheres.

1.4. Dry drug layered pellets in a fluid bed processor (FBP) at 40° C. until loss on drying (LOD) at 60° C. is less than 2% w/w.

2. Subcoating 2.1. Dissolve hydroxypropyl cellulose in a mixture of isopropyl alcohol and methylene chloride.

2.2. Sift talc and titanium dioxide through an ASTM #60 mesh sieve.

2.3. Disperse talc and titanium dioxide in a mixture of isopropyl alcohol and methylene chloride and circulate through a colloid mill.

2.4. Add dispersion of 2.3 to polymer solution of 2.1 and stir.

2.5. Subcoat drug layered pellets from 1.4 using a FBP, and dry.

3, Delayed Release Coating

3.1. Disperse methacrylic acid copolymer type C in water, to form a 30% w/w dispersion.

3.2. Dissolve PEG 6000 in water and add dispersion of 3.1. Disperse talc (first quantity) and titanium dioxide in the dispersion and homogenize for 15 minutes.

3.3, Dissolve polysorbate 80 in warm water and cool.

3.4. Add the solution of 3.3 to the dispersion of 3.2 and stir.

3.5. Spray the dispersion of 3.4 onto subcoated pellets of 2.5 to produce a weight gain of 20% w/w, after drying, using a FBP.

3.6. Dry coated pellets in the FBP until LOD 1-3% w/w at 60° C.

3.7. Cure the pellets in the FBP at 40° C. for 2 hours.

3.8. Add talc (second quantity) to the coated pellets in the FBP and fluidize for 10 minutes.

4. Extended Release Coating

4.1. Dissolve Eudragit S100 and Eudragit L100 in a mixture of isopropyl alcohol and water, then dissolve triethyl citrate in the solution.

4.2. Add talc (first quantity) to solution with continuous stirring.

4.3. Spray the dispersion of 4.2 onto subcoated pellets of 2.5 to produce a weight gain of 40% w/w, after drying, using a FBP.

4.4. Dry coated pellets in the FBP until LOD is 1-3% w/w at 60° C.

4.5. Cure the pellets in the FBP at 40° C. for 2 hours.

4.6. Add talc (second quantity) to the coated pellets in the FBP and fluidize for 10 minutes.

5. Encapsulation

5.1. Fill delayed release pellets of 3.8 containing 25% of the dexlansoprazole dose, and extended release pellets of 4.6 containing 75% of the dexlansoprazole dose, into an empty hard gelatin capsule.

1. A solid premix comprising dexlansoprazole, or a pharmaceutically acceptable salt thereof, and at least one pharmaceutically acceptable excipient, prepared by combining a solution of dexlansoprazole or a salt thereof in an organic solvent with a water soluble excipient, and removing the solvent.

2. (canceled)

3. The solid premix of claim 1, wherein a pharmaceutically acceptable excipient comprises a water soluble sugar excipient.

4. The solid premix of claim 1, wherein a pharmaceutically acceptable excipient comprises a water soluble polymer.

5. The solid premix of claim 1, having a weight ratio of dexlansoprazole to pharmaceutically acceptable excipient from about 2:1 to about 1:10.

6. The solid premix of claim 1, having a weight ratio of dexlansoprazole to pharmaceutically acceptable excipient from about 1:1 to about 1:6.

7. The solid premix of claim 1, having a weight ratio of dexlansoprazole to pharmaceutically acceptable excipient from about 1:1 to about 1:4.

8. The solid premix of claim 1, having a mean particle size less than about 500 μm.

9. The solid premix of claim 1, having a mean particle size less than about 250 μm.

10. A pharmaceutical formulation, comprising a solid premix of claim 1 and at least one additional pharmaceutically acceptable excipient.

11. The pharmaceutical formulation of claim 10, in the form of granules, pellets, spherules, micro tablets, a tablet, a capsule, or a capsule filled with particles.

12. The pharmaceutical formulation of claim 10, in the form of a capsule filled with particles, wherein a particle comprises:
a) a core comprising a solid premix;
b) optionally, a separating layer surrounding the core; and
c) an enteric coating surrounding the core of a) or separating layer of b).

13. The pharmaceutical formulation of claim 12, wherein a core comprises an inert particle having a coating comprising a premix.

14. The pharmaceutical formulation of claim 12, having a separating layer comprising a polymer.

15. The pharmaceutical formulation of claim 12, having a separating layer comprising a cellulose derivative.

16. The pharmaceutical formulation of claim 12, comprising two or more fractions of particles, each fraction being provided with a different enteric coating.

17. The pharmaceutical formulation of claim 12, comprising particles having a delayed release enteric coating and particles having an extended release enteric coating.

18. A method for treating erosive esophagitis and heartburn associated with non-erosive gastroesophageal reflux disease in a mammal, comprising administering a pharmaceutical formulation of claim 10.

19. A process for preparing a solid premix comprising:
a) combining a solution of dexlansoprazole, or a salt thereof, with a water-soluble excipient; and
b) removing solvent.

20. The process of claim 19, wherein a solution comprises dexlansoprazole in an organic solvent.

21. The process of claim 19, wherein a water soluble excipient comprises a sugar excipient.

22. The process of claim 19, wherein a water soluble excipient comprises a polymer.
